(19) United States
(12) Patent Application Publication
(10) Pub. No.: US 2026/0144572 A1
(43) Pub. Date: May 28, 2026

(54) Surgical Staple or Anchor System

(71) Applicant: KiriGenX, Inc., Phoenix, AZ (US)

(72) Inventor: Theodore R. Kucklick, Scotts Valley, CA (US)

(73) Assignee: KiriGenX, Inc., Phoenix, AZ (US)

(21) Appl. No.: 19/400,330

(22) Filed: Nov. 25, 2025

Related U.S. Application Data
(60) Provisional application No. 63/725,122, filed on Nov. 26, 2024.

(51) Int. Cl. A61B 17/34; A61B 17/00; A61B 17/04; A61B 17/064; A61B 17/068

(52) U.S. Cl. CPC A61B 17/3468; A61B 17/00234; A61B 17/068; A61B 2017/00238; A61B 2017/00367; A61B 2017/00991; A61B 2017/0409; A61B 2017/0464; A61B 17/0642

(57) ABSTRACT

A surgical stapler that can deliver staples or anchors into both tendon and bone. The staples or anchors releasably engage to the end of a staple rod. The stapler rod includes a butterfly clip on its proximal end, the butterfly clip having two wings or panels and a striking surface between the wings. The stapler is used to secure a biological construct to tendon or bone during arthroscopic surgery. The device includes a stapler handle with an insertable staple rod. The stapler handle drives the staple rod to advance a staple into the bone or tendon.

This application claims priority to U.S. Provisional Patent Application 63/725,122, filed Oct. 26, 2024.

### FIELD OF INVENTION

The inventions described below relate to the field of surgical fastening devices, and more particularly to surgical stapler systems configured to deliver staples or anchors through a minimally invasive pathway.

### BACKGROUND OF THE INVENTIONS

Surgical procedures commonly require the fixation or anchoring of soft tissue, bone, or biological constructs. To accomplish these tasks, various surgical staplers have been developed. Conventional surgical staplers typically incorporate an internal staple cartridge or magazine pre-loaded with multiple staples. Activation of such devices often relies on complex handle mechanics that actuate a driver or push rod to deform the staple against an anvil or opposing surface positioned at the distal end of the instrument.

Minimally invasive and arthroscopic procedures have created demand for stapling instruments that are more maneuverable and capable of precise placement within narrow operative corridors.

Existing staple drivers often lack modularity. Because the staple and driver components are typically integrated into a single cartridge or jaw assembly, the entire instrument or a substantial portion of the end-effector must be replaced after deployment. This increases waste and cost, and may limit the surgeon's ability to selectively load specific fasteners during a procedure. There remains a need for improved stapler systems that simplify the mechanical activation pathway and allow individual staples or clips to be loaded on demand. There is a further need for or systems that accommodate removable or replaceable staple rods, provide reliable delivery through a narrow cannula or portal, and maintain consistent deployment force while reducing overall device complexity.

### SUMMARY

The devices and methods described below provide for a surgical stapler that can deliver staples or anchors into both tendon and bone where both the staples or anchors lock into a common interface. The stapler is used to secure a biological construct or implant into tendon or bone during arthroscopic surgery. The system includes a handle, a trigger, a cannula tube extending distally from the handle, and an insertable staple or anchor rod configured to receive and support a bone or tendon staple. The staple or anchor rod may include a clip, butterfly clip or similar retention mechanism that enables the rod to be secured within, and selectively released from, the handle.

Further actuation of the trigger drives the staple or anchor into tissue, and, when inserting the staple or anchor into bone, a mallet may be used to tap the proximal striking face of the clip to ensure complete seating of the staple or anchor. Upon full insertion, the trigger is released to disengage the staple from the staple or anchor rod. The clip may then be compressed to release the staple or anchor rod from the handle, enabling removal and replacement with a new pre-loaded rod. Additional staples or anchors may be delivered in the same manner until the patch or construct is fully fixated. The system provides a controlled, repeatable, and minimally invasive technique for delivering staples or anchors into bone or soft tissue, improving fixation accuracy while reducing procedural complexity.

### BRIEF DESCRIPTION OF THE DRAWINGS

FIG. 1 is an exploded view of a stapler system including a handle, slider block, staple or anchor rod and butterfly clip.

FIG. 2 is a view of the stapler system where the staple or anchor rod is releasably received through a proximal opening of the handle.

FIG. 3a is a front view of the stapler system and FIG. 3b is a cross sectional view of FIG. 3a along line A-A.

FIGS. 4a and 4b show the anchor trocar tip of the staple or anchor rod and FIG. 4c is a cross-sectional view of FIG. 4a.

FIG. 5 shows an exploded view of an alternative staple system including a handle, slide block, staple rod and single wing clip.

FIG. 6 shows the alternative staple system of FIG. 5 where the staple or anchor rod is releasably received through a proximal opening of the handle.

### DETAILED DESCRIPTION OF THE INVENTIONS

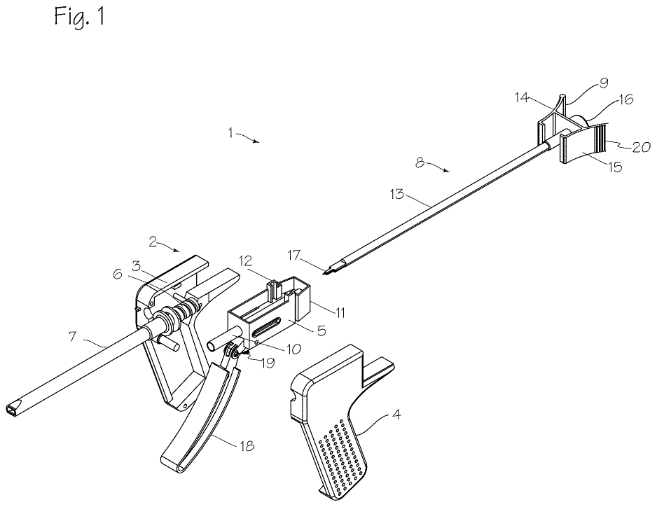

FIG. 1 illustrates a stapler system 1 including a handle 2 having first housing portion 3 and second mating housing portion 4 with an inner longitudinal lumen or chamber that extends parallel to the length of the handle, a slider block 5 disposed within the inner longitudinal chamber, a spring 6, a cannula 7, and an insertable staple or anchor rod 8 having a butterfly clip 9 at the distal end of the staple or anchor rod. The slider block has a spring mount within a telescoping tube 10 at the distal end of the slider block. The slider block further includes at least one engagement tab or groove 11 at a proximal end of the slider block configured to releasably engage the staple or anchor rod and at least two upwardly projecting tabs 12 configured to stabilize the slider block against the handle when the slider block is driven forward. The slider block includes longitudinal bore extending through the distal portion for receiving the trocar tip of the proximal end of the stapler rod. The engagement tab connects the slider block to the trigger for actuation by a user. The spring mount is positioned proximal to the slider block and adjacent to the compression spring. The telescoping tube or distal guide sleeve is integrated at the distal end to create a stable lumen for the stapler rod trocar piercing tip. The spring 6 is sized and dimensioned to be positioned over the telescoping tube and spring mount at the distal end of the handle and arranged to bias the slider block toward a proximal position after actuation. The spring is loaded immediately adjacent to the spring mount so that the spring axis is collinear with the slider block travel axis to ensure linear force delivery. The cannula 7 is positioned adjacent to the spring and projects from the distal end of the handle. The staple or anchor rod 8 is configured to be received within and advanced through the inner longitudinal chamber of the cannula. The staple or anchor rod includes an implant holder shaft 13 having a proximal end and a distal end, a butterfly clip 9 including two wings or panels 14, 15 and a striking face 16 between the two panels, the butterfly clip is disposed at the proximal end of the implant holder shaft and at least one staple or anchor supported at the distal end of the implant holder shaft. The staple or anchor rod can transmit forward force to the legs of a staple or engage an anchor head, anchor tail, or insertion interface. The staple or anchor rod further includes a trocar tip 17 positioned at the distal end of the implant holder shaft and configured to releasably secure the at least one staple or anchor. The trocar tip also includes a central anvil 18 configured to maintain alignment of the at least one staple or anchor prior to deployment. A trigger 19 is also mounted via a pivot 20 to the slider block. The trigger is operably coupled to the slider block such that pivoting of the trigger relative to the handle axially advances or retracts the staple rod within the inner longitudinal chamber of the handle. When the staple or anchor rod is inserted within the stapler, the staple or anchor rod is advanced through the cannula and the staple or anchor of the staple or anchor rod is stored within the cannula until it is inserted into the desired placement position. Then the staple or anchor rod is advanced to project through the cannula to be driven into the tendon or bone.

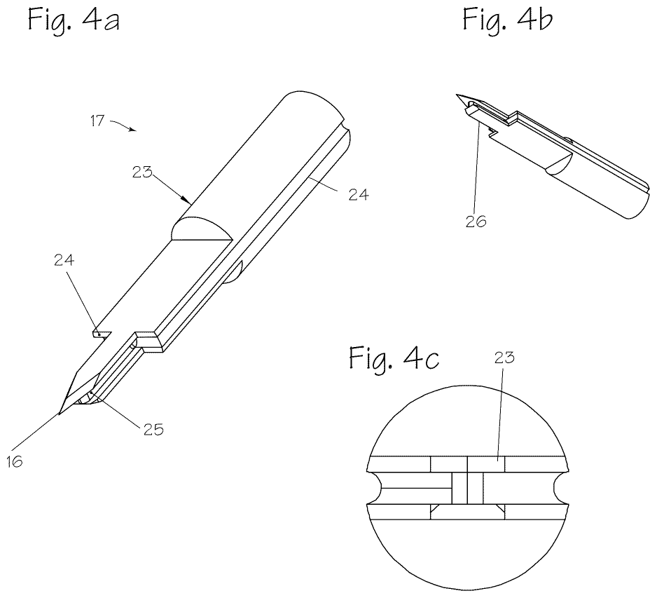

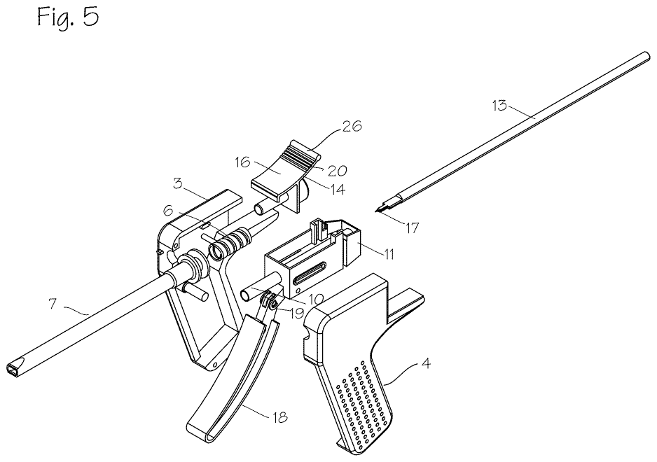

The staple or anchor rod includes an implant holder shaft 13 having a proximal and distal end. The proximal end includes the butterfly clip 9 and the distal end includes at least one staple or anchor supported on a trocar tip 17 (shown in FIGS. 4a and 4b). The butterfly clip includes two thin, symmetrically arranged wings or panels 14, 15 that extend laterally from the rod central body 13. Alternatively, the butterfly clip can include only a single wing as shown in FIG. 5. Each wing can include outwardly tapering panels that have staple or anchor support grooves. The wings open and close to adjust the position of the rod within the slider block of the stapler. The butterfly clip can be made of any biocompatible, sterilizable resilient material such as acrylonitrile butadiene styrene (ABS), polycarbonate, nylon or stainless steel. The butterfly clip can be releasably coupled to the distal end of the staple rod or anchor rod by a detent feature or snap-fit interface. The butterfly clip releasably engages the clip and rod assembly to the slider block. The wings distribute axial load from the rod so that staple motion is uniform. The wings also prevent tilting or asymmetrical driving of the staple. The wings can include gripping ridges 21 on an end for secure engagement by a user. The trocar tip releasably secures either a tendon or bone staple or anchor. The staples or anchors are preloaded around a central anvil 22 housed within the driver. The central anvil keeps the staples or anchors aligned and supported until the trigger is pulled to actuate the trocar tip to drive the staples or anchors into the predetermined target tissue. The trocar tip pierces the implant and penetrates either the tendon or bone to deliver an anchor to the predetermined location, making external sharp prongs on the cannula shaft tip unnecessary. The trocar tip can be concealed within the cannula for protection until inserted into the desired location within a patient and without requiring an additional cannula. The proximal end of the trocar tip inserts into the distal telescoping tube of the slider block. When the trigger 19 is actuated, the slider block 5 translates distally guided by the spring mount within the telescopic tube 10. The slider block applies an axial force to the trocar tip and the trocar tip enters the cannula tube. When the trocar tip reaches the distal end, it contacts the anvil to deliver and shape the staple or anchor. The spring mounted on the slider block then pushes the slider block proximally and the trocar tip follows until fully retracted and returned to a position for loading another staple or anchor. A desired tendon or bone staple is preloaded onto the distal end of the staple rod and can include a single staple or multiple staples in a cartridge. The butterfly clip includes a striking face 16 configured for a surgeon to strike the face of the butterfly clip with a mallet or other tool to drive the staple into bone. When the trigger is pressed, force is transferred to the staple or anchor rod which is driven forward and drives the staple or anchor from the distal end of the staple or anchor rod into the desired tendon or bone. The butterfly clip can also include tangs that releasably engage to the vertical engagement tab or groove on the slider block. The staple or anchor rod is advanced linearly within the cannula and as the staple or anchor rod moves forward to drive the staple, the spring returns the rod to the original position after deployment. Staples or anchors are preloaded onto the staple or anchor rod at the distal end of the rod. The preloaded rod is inserted into the housing and advanced through the handle so that the loaded staple or anchor projects from the distal end of the handle.

The butterfly clip at the proximal end of the staple or anchor rod is releasably secured to the distal end of the rod. The surgical stapler is positioned at the desired location and the trigger is pulled so that the rod is moved forward and the staple or anchor is delivered into the desired tendon or bone. The slider block is the movable component that slides within the inner longitudinal chamber. The slider block is positioned next to the spring that exerts force to resist movement or return the slider block to a default position when force is released. As the slider block moves, the spring compresses or stretches, providing resistance proportional to the displacement.

The trigger initiates the firing mechanism that releases staple or anchor and advances it to the tendon or bone. The trigger allows for single or multiple firings. The spring stores energy when trigger is pulled. The trigger acts upon the spring to drive the staple or anchor into tendon or bone. When the trigger is pressed, the slider block moves in conjunction with the spring and pushes the staples or anchors into the tendon or bone. After firing, the slider block is returned to original position to reset for next firing. When the staple or anchor is driven into the tendon or bone, the spring returns to its initial position for next use. When the trigger is pulled, it acts on slider block so that the spring's stored energy pushes or slides the slider block back to its original position. The spring mount is fixed at one end of the slider block and attached to the spring. When the trigger is pulled, the spring is compressed or extended. Pulling the trigger advances the slider block, compressing or extending the spring. The spring mount anchors the spring in place, allowing the spring to store potential energy. Pulling the trigger moves the slider block to actuate the trocar tip to release the staple into the target tissue. The spring advances the staple from the trocar tip to the targeted tissue. When the trigger is released, spring's force pushes slider block back to original position. Once the staple or anchor is deployed from the slide, the trigger retracts the rod back into the cannula tube. The staple or anchor rod is unclipped and removed and the next staple is loaded onto the staple or anchor rod.

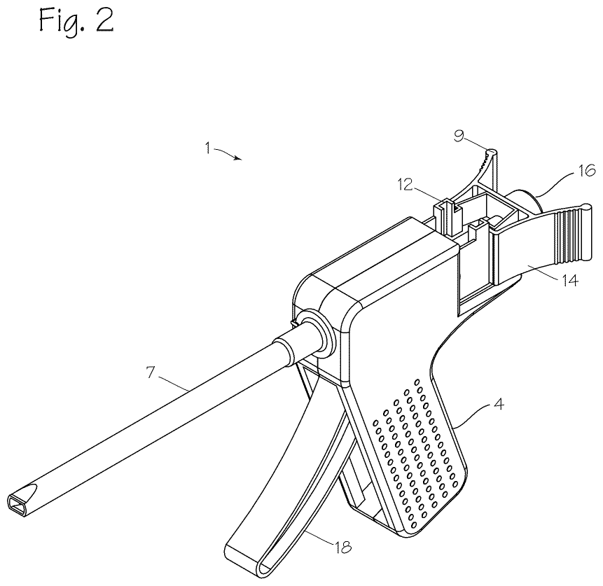

FIG. 2 is a view of the stapler system where the staple or anchor rod 8 is releasably received through a proximal opening of the handle 2. The staple or anchor rod is elongate and sized to pass through the longitudinal chamber and engage the slider block. The proximal end of the stapler or anchor rod 8 includes the butterfly clip 9 with the wings 14, 15 and striking face 16 for the user to strike the striking face when inserting the staple or anchor into bone. When the staple or anchor rod is inserted into the handle, the distal end of the staple or anchor rod contacts the engagement tab of the slider block. Continued forward advancement of the staple or anchor rod causes the engagement tab to be driven forward, shifting the slider block distally within the longitudinal chamber. As the slider block advances, the telescoping distal tube of the slider block moves forward into the cannula tube to deliver the staple into an ejection position at the distal end of the cannula tube. The biasing spring is compressed during this advancement, storing energy for automatic return of the slider block upon withdrawal of the staple or anchor rod.

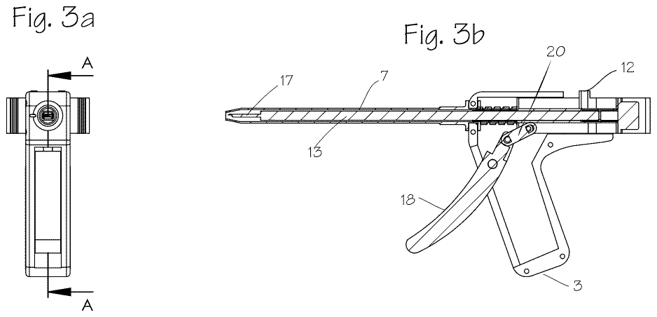

FIG. 3a is a front view of the stapler system and FIG. 3b is a cross-sectional view of FIG. 3a along line A-A. This figure illustrates the staple or anchor rod 8 advanced through the slider block and into the cannula 7. The trocar piercing tip 17 projects from the distal end of the staple or anchor rod within the cannula. The staple or anchor is also positioned on the distal end of the stapler or anchor rod within the cannula. Pulling the trigger advances the slider block forward to drive the staple or anchor into the desired location. Release of the trigger retracts the stapler or anchor rod back into the cannula tube for removal of the stapler or anchor rod and reloading of additional staples or anchors.

FIGS. 4a and 4b show the anchor trocar tip 17 of the staple or anchor rod and FIG. 4c is a cross-sectional view of FIG. 4a. The trocar tip pierces an implant and penetrates tendon or bone to deliver the staple or anchor. It includes a monorail I-beam driver 23 (shown in cross section in FIG. 4c) terminating in a trocar pilot tip. The driver tapers to the tipped end and includes an intermediate shoulder stop 24 and also includes a suture groove 25 within a central channel portion of the driver. The suture grooves provide for friction or mechanical retention of the staples or anchors, preventing them from slipping out prematurely. They also assist in aligning the staples or anchors for consistent placement. The central anvil 22 projects from the monorail I-beam between the anchor trocar tip point and a lower beam rail as shown in FIG. 4b. All staples or anchors wrap around the center anvil and fit within the side grooves. The anchor tip can fit any shape staple or anchor. The I-beam driver delivers the staple or anchor through the biological implant or construct sheet to be affixed to the tendon or bone. The trocar pilot tip makes a hole through the construct sheet so it does not bunch up when pushing the hard staple or anchor through the sheet. The I-beam driver supports a variety of anchors and staples. Including barbed, flukes, wings and all suture.

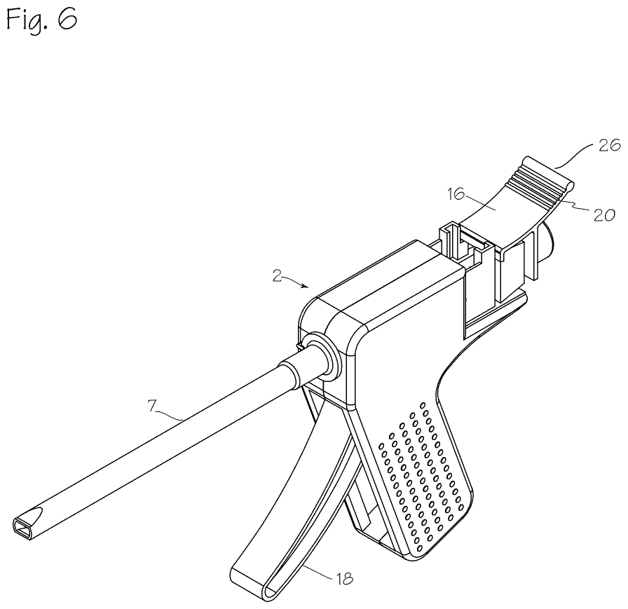

FIG. 5 shows an exploded view of an alternative staple system including a stapler handle, slide block, staple rod and single wing clip. The stapler system 1 includes a handle 2 having first housing portion 3 and second mating housing portion with an inner longitudinal lumen or chamber that extends parallel to the length of the handle, a slider block 5 disposed within the inner longitudinal chamber, a spring 6, a cannula 7, and an insertable staple or anchor rod 8 having a single panel clip 26 at the distal end of the staple or anchor rod. The slider block has a spring mount within a telescoping tube 10 at the distal end of the slider block. The slider block further includes at least one engagement tab or groove 11 at a proximal end of the slider block configured to releasably engage the staple rod and at least two upwardly projecting tabs 12 configured to stabilize the slider block against the handle when the slider block is driven forward. FIG. 6 shows the alternative staple system of FIG. 5 where the staple or anchor rod is releasably received through a proximal opening of the handle. The clip includes a single panel 14 with griping ridges 20 and a striking face 16.

In use, a bone or tendon staple or anchor is loaded onto the staple or anchor rod including a single wing or butterfly clip and the staple or anchor rod is inserted into the handle until the staple or anchor is positioned at the distal end of the cannula tube of the handle. Once the staple or anchor rod is pushed into the correct forward position, it clicks onto the cannula tube of the handle. Axial alignment of the single wing or butterfly clip is achieved by constraining lateral movement of the clip. The single wing or butterfly clip is locked onto the staple or anchor rod prior to inserting the staple or anchor rod into the stapler. The surgeon then establishes an arthroscopic portal. The surgeon then inserts the driver cannula into the arthroscopic portal and places the tip of the staple or anchor rod with the staple at a predetermined patch fixation location. The trigger is pulled to partially expose the trocar tip in order to manipulate the patch or construct into the desired location within the patient. The staple or anchor rod tip is retracted and the cannula tip is pressed against the predetermined fixation location. The trigger is pulled while exerting firm pressure. If the staple or anchor is inserted into bone, a mallet is used to tap the striking face of the staple or anchor rod to further drive the anchor into the bone. When the staple or anchor is fully seated, the trigger is released and pulled back to release the staple or anchor. Deployment of the staple or anchor includes using distal edges of the wings of the clip to position or stabilize tissue immediately prior to staple penetration. The single or double wings of butterfly clip are then squeezed or compressed to release the staple or anchor rod from the handle and removed and another staple or anchor is inserted into the staple or anchor rod and pushed forward to lock into place. Additional staples or anchors are inserted in the same manner until the patch or construct is fixated in place. After pulling the trigger, the striking face of the clip can optionally be tapped with a mallet to further drive the staple into the bone.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

We claim:

1. A surgical stapler system comprising:
a handle having first housing portion and second mating housing portion with an inner longitudinal chamber that extends parallel to the length of the handle;
a slider block disposed within the inner longitudinal chamber, the slider block including a telescoping tube at a distal end of the slider block, a spring mount positioned within the telescoping tube, at least one engagement tab or groove at a proximal end of the slider block configured to releasably engage a staple rod and at least two upwardly projecting tabs configured to stabilize the slider block against the handle when the slider block is driven forward;
a spring sized and dimensioned to be positioned over the telescoping tube and spring mount at the distal end of the handle and arranged to bias the slider block toward a distal position after actuation;
a cannula positioned adjacent to the spring and projecting distally from the distal end of the handle;
an insertable rod configured to be received within and advanced through the inner longitudinal chamber and the cannula, the rod including an implant holder shaft having a proximal end and a distal end, a clip including at least one panel and a striking face adjacent to the panel, the clip disposed at the proximal end of the implant holder shaft and at least one anchor supported at the distal end of the implant holder shaft; and a trocar tip positioned at the distal end of the implant holder shaft and configured to releasably secure the at least one anchor, the trocar tip including a central anvil configured to maintain alignment of the at least one anchor prior to deployment; and
a trigger pivotably mounted to the slider block;
wherein the trigger is operably coupled to the slider block such that pivoting of the trigger relative to the handle axially advances or retracts the rod within the inner longitudinal chamber of the handle.

2. The surgical stapling system of claim 1, wherein the clip further includes a second panel.

3. The surgical stapling system of claim 2, wherein each panel of the clip has a distal guide edge configured to align an anchor during deployment.

4. The surgical stapling system of claim 2, wherein the clip is releasably coupled to the distal end of the staple rod by a detent feature or snap-fit interface.

5. The surgical stapling system of claim 2, wherein the slider block includes a distal coupling lumen that receives a proximal segment of the rod to maintain axial alignment during advancement.

6. The surgical stapling system of claim 1, wherein the clip is formed of a material selected from the group consisting of acrylonitrile butadiene styrene (ABS), polycarbonate, nylon and stainless steel.

7. The surgical stapling system of claim 2, wherein the cannula tube provides lateral constraint to the clip such that the panels travel coaxially and abut the handle during deployment.

8. The surgical stapling system of claim 2, wherein the clip includes an anchor-support surface configured to contact a proximal portion of an anchor during advancement toward the tissue.

9. The surgical stapling system of claim 2, wherein the rod includes a distal mounting recess or slot that mechanically receives a portion of the clip to secure the clip to the rod.

10. The surgical stapling system of claim 2, wherein the clip panels include inwardly-facing guide surfaces arranged to align the anchor in a predetermined orientation relative to the tissue.

11. A method of implanting a bone or tendon anchor using a surgical stapling system, the method comprising the steps of:
loading a bone or tendon anchor onto the distal end of a rod, the rod including:
an implant holder shaft having a proximal end and a distal end, a clip including at least one panel and a striking face adjacent to the panel, the clip disposed at the proximal end of the implant holder shaft and at least one anchor supported at the distal end of the implant holder shaft, and a trocar tip positioned at the distal end of the implant holder shaft and configured to releasably secure the at least one anchor, the trocar tip including a central anvil configured to maintain alignment of the at least one anchor prior to deployment;
advancing the rod to a forward position within the cannula tube;
establishing an arthroscopic portal in a patient;
inserting a driver cannula of the stapling system through the arthroscopic portal;
positioning the distal tip of the rod and place the anchor at a predetermined patch or construct fixation location;
pulling the trigger to partially expose the trocar tip to manipulate the patch or construct within the patient;
retracting the distal tip of the rod;
pressing the distal tip of the cannula tube against the predetermined fixation location;
pulling the trigger to exert pressure on the handle to drive the anchor into tissue;
releasing the trigger and disengage the anchor from the rod;
compressing the panel of the clip to release the rod from the handle; and
removing the rod from the handle.

12. The method of claim 11 wherein after pulling the trigger, the striking face of the clip is tapped with a mallet to further drive the anchor into the bone.

13. The method of claim 11 wherein advancing the clip distally further comprises the step of maintaining axial alignment of the clip by constraining lateral movement of the clip.

14. The method of claim 11 wherein the method further comprises the step of locking the clip onto the rod prior to inserting the rod into the stapler system.

15. The method of claim 11 wherein deploying the anchor includes using distal edges of the wing of the clip to position or stabilize tissue immediately prior to anchor penetration.
